(19) United States
(12) Patent Application Publication
(10) Pub. No.: US 2006/0002508 A1
(43) Pub. Date: Jan. 5, 2006

(54) X-ray CT apparatus

(75) Inventor: Mitsuru Yahata, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/169,373

(22) Filed: Jun. 29, 2005

(30) Foreign Application Priority Data
Jul. 5, 2004 (JP) 2004-197912

(51) Int. Cl. A61B 6/00; G01N 23/00

(52) U.S. Cl. 378/19

(57) ABSTRACT

An X-ray CT apparatus that includes a multidetector wherein a scintillator unfractionated by reflectors or slits or the like is laminated on an upper surface of a photodiode array comprising photodiodes two-dimensionally arranged in channel and slice directions, a DAS which acquires signals delivered from the photodiodes, and a signal transfer section which switches whether to transfer the signals sent from the respective ones of the photodiodes to the DAS or to add the signals sent from the 2×2 photodiodes of the photodiodes and transfer the result of addition to the DAS.

### BACKGROUND OF THE INVENTION

The present invention relates to an X-ray CT apparatus, and more specifically to an X-ray CT apparatus capable of performing high resolution photography.

In order to enable an X-ray CT apparatus to carry out high resolution photography, there have heretofore been proposed an X-ray detector (refer to, for example, the following patent document 1) wherein a plurality of photodiodes are provided every cells fractionated by collimators, an X-ray detector (refer to, for example, the following patent document 2) wherein reflectors which divide a scintillator into a large number of cells, are tilted, etc.

[Patent Document 1] Japanese Unexamined Patent Publication No. 2004-93489

[Patent Document 2] Japanese Unexamined Patent Publication No. 2004-28815

The conventional X-ray CT apparatus has the following problems.

(1) Although the X-ray CT apparatus has the merits of obtaining a high resolution image if high resolution photography is performed, it has also the demerits of increasing a burden on signal processing, narrowing a photography range so long as an increase in the number of photodiodes is not made, for example. That is, if the high resolution photography is made even to an application enough at a low resolution image, then only the demerits exist.

(2) In the conventional X-ray detector, a scintillator has been fractionated by reflectors or slits in order to prevent even photodiodes adjacent to each other from receiving light to be light-received by a given photodiode alone. However, the existence of the reflectors or slits or the like reduces the efficiency of light emission of the scintillator. Although the reduction in luminous or light emission efficiency has heretofore been accepted, the reduction in luminous efficiency cannot be accepted where resolution is enhanced.

(3) In the conventional X-ray detector, collimators have been placed on a scintillator to compart or block out cells. However, the existence of the collimators reduces the efficiency of light emission of the scintillator. Although the reduction in the light-emission efficiency has heretofore been accepted, the reduction in the light-emission efficiency cannot be accepted where resolution is enhanced.

(4) In the conventional X-ray detector, an X-ray shield extending in a channel direction has been placed on a scintillator to prevent interference between cells as viewed in a slice direction. However, the existence of the X-ray shield reduces the efficiency of light emission of the scintillator. Although the reduction in light-emission efficiency has heretofore been accepted, the reduction in light-emission efficiency cannot be accepted where resolution is enhanced.

### SUMMARY OF THE INVENTION

Therefore, an object of the present invention is to provide an X-ray CT apparatus capable of performing high resolution photography.

In a first aspect, the present invention provides an X-ray CT apparatus comprising an X-ray tube, an X-ray detector in which an unfractionated scintillator is laminated on an upper surface of a photodiode array comprising photodiodes two-dimensionally arranged in a channel direction and a slice direction, a DAS which acquires signals delivered from the photodiodes, and signal switching means which switches whether to transfer the signals sent from the respective ones of the photodiodes to the DAS or to add the signals sent from the N×N (where N: integer greater than or equal to 2) photodiodes of the photodiodes and transfer the result of addition to the DAS.

In the X-ray CT apparatus according to the first aspect, the signals sent from the respective ones of the photodiodes are transferred to the DAS in the case of an application which requires a high resolution image. In an application enough at a low resolution image, the signals sent from the N×N (where N: integer greater than or equal to 2) photodiodes of the photodiodes are added and the result of addition is transferred to the DAS. If the photography ranges at the high resolution photography and low resolution photography are nearly equal, then the number of signals can be reduced, and a burden on the signal processing at the low resolution photography can be lessened. On the other hand, if the burden on signal processing may be the same degree, then photodiodes used upon the low resolution photography can be added, thus making it possible to extend a photography range.

Incidentally, the term “unfractionated scintillator” in the above configuration indicates a scintillator unfractionated into a large number of cells by reflectors or slits or the like. In order to prevent a photodiode of a given cell from receiving light of a cell adjacent thereto, scintillators have heretofore been fractionated every cells by the reflectors or slits or the like. However, the existence of the reflectors or slits or the like reduces the efficiency of light emission of the scintillator. Although the reduction in light-emission efficiency could be accepted when the pitch of each photodiode in the photodiode array was increased (to e.g., 1.0 mm), the reduction in light-emission efficiency cannot be accepted when the pitch of the photodiode is reduced (to e.g., 0.5 mm). Thus, the unfractionated scintillator was adopted in the X-ray CT apparatus according to the first aspect. Thinning the scintillator (to, e.g., 1 mm or less) in conformity with the reduction in the pitch of the photodiode makes it possible to restrain a photodiode adjacent to a given photodiode from receiving light to be received or detected by the given photodiode.

In a second aspect, the present invention provides an X-ray CT apparatus wherein in the X-ray CT apparatus according to the first aspect, the photodiode array includes a high resolution block having a pitch extending in each of the channel and slice directions Ph≦0.6 mm, and low resolution blocks each having a pitch extending in each of the channel and slice directions Pl=N×Ph, and when the number of the photodiodes in the channel direction in the high resolution block is Ch, the number of photodiodes in the slice direction is Sh, the number of the photodiodes in the channel direction in each of the low resolution blocks is Cl, the number of the photodiodes in the slice direction is Sl, and the number D of signals inputtable to the DAS is D, the following relationship is established:
D=Ch×Sh=Ch×Sh/(N×N)+Cl×Sl

Since the number of signals inputted to the DAS is a constant number D in the X-ray CT apparatus according to the second aspect, burdens on signal processing at high resolution photography and low resolution photography are nearly equal. Since, however, the photodiodes of the low resolution block can also be added and used upon the low resolution photography, a photography range can be extended.

In a third aspect, the present invention provides an X-ray CT apparatus comprising an X-ray tube, a high-resolution X-ray detector in which an unfractionated scintillator is laminated on an upper surface of a photodiode array comprising photodiodes two-dimensionally arranged with a pitch Ph<0.6mm in each of channel and slice directions, a low-resolution X-ray detector in which a scintillator is laminated on an upper surface of a photodiode array comprising photodiodes two-dimensionally arranged with a pitch Pl>Ph in the channel and slice directions, a DAS which acquires signals from the photodiodes, and signal switching means which switches whether to transfer the signals from the photodiodes of the high-resolution X-ray detector to the DAS or to transfer the signals from the photodiodes of the low-resolution X-ray detector to the DAS.

In the X-ray CT apparatus according to the third aspect, the signals sent from the high-resolution X-ray detector are transferred to the DAS in an application which requires a high resolution image. In an application enough at a low resolution image, the signals sent from the low-resolution X-ray detector are transferred to the DAS. If photography ranges at high resolution photography and low resolution photography are of the same degree, then the number of signals can be reduced, and a burden on signal processing at the low resolution photography can be reduced. On the other hand, if the burdens on the signal processing may be the same degree, then the photography range at the low resolution photography can be extended.

Incidentally, the term “unfractionated scintillator” in the above configuration indicates a scintillator unfractionated into a large number of cells by reflectors or slits or the like. In order to prevent a photodiode of a given cell from receiving light of a cell adjacent thereto, scintillators have heretofore been fractionated every cells by the reflectors or slits or the like. However, the existence of the reflectors or slits or the like reduces the efficiency of light emission of the scintillator. Although the reduction in light-emission efficiency could be accepted when the pitch of each photodiode in the photodiode array was increased (to e.g., 1.0 mm), the reduction in light-emission efficiency cannot be accepted when the pitch of the photodiode is reduced (to e.g., 0.5 mm). Thus, the unfractionated scintillator was adopted in the X-ray CT apparatus according to the third aspect. Thinning the scintillator (to, e.g., 1 mm or less) in conformity with the reduction in the pitch of the photodiode makes it possible to restrain a photodiode adjacent to a given photodiode from receiving light to be received or detected by the given photodiode.

In a fourth aspect, the present invention provides an X-ray CT apparatus comprising an X-ray tube, a high-resolution X-ray detector in which an unfractionated scintillator is laminated on an upper surface of a photodiode array comprising photodiodes two-dimensionally arranged with a pitch Pl≦0.6 mm in channel and slice directions, a low-resolution X-ray detector in which a scintillator is laminated on an upper surface of a photodiode array comprising photodiodes two-dimensionally arranged with a pitch Pl>Ph in the channel and slice directions, a DAS which acquires signals from the photodiodes, signal adding means which adds the signals sent from the photodiodes of the high-resolution X-ray detector and the signals sent from the photodiodes of the low-resolution X-ray detector and transfers the result of addition to the DAS, and X-ray adjusting means which switches whether to launch an X-ray into the high-resolution X-ray detector alone or to launch an X-ray into the low-resolution X-ray detector alone.

In the X-ray CT apparatus according to the fourth aspect, the X-ray is launched into the high resolution X-ray detector alone in an application which requires a high resolution image, and the X-ray is launched into the low resolution X-ray detector in an application enough at a low resolution image. If photography ranges for the high-resolution X-ray detector and the low-resolution X-ray detector are of the same degree, then the number of signals can be reduced, and a burden on signal processing at low resolution photography can be reduced. On the other hand, if the burdens on the signal processing may be the same degree, then the photography range at the low-resolution X-ray detector can be extended.

Incidentally, the term “unfractionated scintillator” in the above configuration indicates a scintillator unfractionated into a large number of cells by reflectors or slits or the like. In order to prevent a photodiode of a given cell from receiving light of a cell adjacent thereto, scintillators have heretofore been fractionated every cells by the reflectors or slits or the like. However, the existence of the reflectors or slits or the like reduces the efficiency of light emission of the scintillator. Although the reduction in light-emission efficiency could be accepted when the pitch of each photodiode in the photodiode array was increased (to e.g., 1.0 mm), the reduction in light-emission efficiency cannot be accepted when the pitch of the photodiode is reduced (to e.g., 0.5 mm). Thus, the unfractionated scintillator was adopted in the X-ray CT apparatus according to the fourth aspect. Thinning the scintillator (to, e.g., 1 mm or less) in conformity with the reduction in the pitch of the photodiode makes it possible to restrain a photodiode adjacent to a given photodiode from receiving light to be received or detected by the given photodiode.

In a fifth aspect, the present invention provides an X-ray CT apparatus wherein in the X-ray CT apparatus of the above configuration, when the number of the photodiodes lying in the channel direction in the high-resolution X-ray detector is Ch, the number of the photodiodes lying in the slice direction in the high-resolution X-ray detector is Sh, and the number of the photodiodes lying in the channel direction in the low-resolution X-ray detector is Cl and the number of the photodiodes lying in the slice direction in the low-resolution X-ray detector is Sl, and the number of the signals inputtable to the DAS is D, the following relationship is established:
D=Ch×Sh=Cl×Sl

Since the number of signals inputted to the DAS is a constant number D in the X-ray CT apparatus according to the fifth aspect, burdens on signal processing at high resolution photography and low resolution photography are nearly equal. However, the photography range of the low resolution X-ray detector can be extended.

In a sixth aspect, the present invention provides an X-ray CT apparatus comprising an X-ray tube, and an X-ray detector in which a nonfractionated scintillator is laminated on an upper surface of a photodiode array comprising photodiodes two-dimensionally arranged in channel and slice directions.

Term “unfractionated scintillator” in the above configuration indicates a scintillator unfractionated into a large number of cells by reflectors or slits or the like. In order to prevent a photodiode of a given cell from receiving light of a cell adjacent thereto, scintillators have heretofore been fractionated every cells by the reflectors or slits or the like. However, the existence of the reflectors or slits or the like reduces the efficiency of light emission of the scintillator. Although the reduction in light-emission efficiency could be accepted when the pitch of each photodiode in the photodiode array was increased (to e.g., 1.0 mm), the reduction in light-emission efficiency cannot be accepted when the pitch of the photodiode is reduced (to e.g., 0.5 mm).

Therefore, the unfractionated scintillator was adopted in the X-ray CT apparatus according to the sixth aspect. Thus, the pitch of each photodiode in the photodiode array can be reduced (to, e.g., 0.6 mm or less).

Thinning the scintillator (to, e.g., 1 mm or less) in conformity with the reduction in the pitch of the photodiode makes it possible to restrain a photodiode adjacent to a given photodiode from receiving light to be received or detected by the given photodiode.

In a seventh aspect, the present invention provides an X-ray CT apparatus wherein in the X-ray CT apparatus of the above configuration, the thickness of the scintillator is less than or equal to 1 mm.

When the “unfractionated scintillator” is used, there is a high possibility that a photodiode adjacent to a given photodiode will receive light to be detected or received by the given photodiode, as compared with the scintillators fractionated by the reflectors.

Therefore, the scintillator was thinned to 1 mm or less in the X-ray CT apparatus according to the seventh aspect. Thus, the light to be received by the given photodiode is launched into its corresponding light-receiving surface of the photodiode at a small incident angle with the angle of incidence 0° as the center, whereas the light is launched into the light-receiving surface of the adjacent photodiode at a large incident angle, whereby interference can be suppressed.

In an eighth aspect, the present invention provides an X-ray CT apparatus wherein in the X-ray CT apparatus according to the sixth aspect, the X-ray detector has collimators which extend in the slice direction on the scintillator at intervals of plural channel skips.

The collimators have heretofore been placed on the scintillator to compart or block out the cells. However, the existence of the collimators reduces the efficiency of light emission of the scintillator. Although the reduction in the light-emission efficiency could be accepted when the pitch of each photodiode in the photodiode array was large (e.g., 1.0 mm), the reduction in the light-emission efficiency cannot be accepted where the pitch of the photodiode is made small (e.g., 0.5 mm).

Therefore, the collimators extending in the slice direction at the intervals of plural channel skips have been adopted in the X-ray CT apparatus according to the eighth aspect. Thus, since it is possible to suppress a reduction in luminous efficiency due to each collimator, the pitch of each photodiode in the photodiode array can be reduced (to, e.g., 0.6 mm or less).

Thinning the scintillator (to, e.g., 1 mm or less) in conformity with the reduction in the pitch of the photodiode makes it possible to restrain a photodiode adjacent to a given photodiode from receiving light to be received or detected by the given photodiode. Thus, no problem occurs even if the collimators extending in the slice direction are provided at the intervals of plural channel skips.

In a ninth aspect, the present invention provides an X-ray CT apparatus wherein in the X-ray CT apparatus according to the sixth aspect, the X-ray detector is not provided with an X-ray shield extending in the channel direction on the scintillator.

The X-ray shield extending in the channel direction has heretofore been placed on the scintillator to prevent interference between the cells as viewed in the slice direction. However, the existence of the X-ray shield reduces the efficiency of light emission of the scintillator. Although the reduction in the light-emission efficiency could be accepted when the pitch of each photodiode in the photodiode array was large (e.g., 1.0 mm), the reduction in the light-emission efficiency cannot be accepted where the pitch of the photodiode is reduced (to e.g., 0.5 mm).

Therefore, the X-ray shield extending in the channel direction is not disposed in the X-ray CT apparatus according to the ninth aspect. Thus, since it is possible to suppress a reduction in luminous efficiency due to the X-ray shield, the pitch of each photodiode in the photodiode array can be reduced (to, e.g., 0.6 mm or less).

Thinning the scintillator (to, e.g., 1 mm or less) in conformity with the reduction in the pitch of the photodiode makes it possible to restrain a photodiode adjacent to a given photodiode from receiving light to be received or detected by the given photodiode. Thus, no problem occurs even if the X-ray shield is discarded.

In a tenth aspect, the present invention provides an X-ray CT apparatus wherein in the X-ray CT apparatus according to the eighth aspect, the X-ray detector is not provided with an X-ray shield extending in the channel direction on the scintillator.

In the X-ray CT apparatus according to the tenth aspect, it is possible to further sufficiently suppress a reduction in luminous efficiency owing to the synergy between the action by the eighth aspect and the action by the ninth aspect.

In an eleventh aspect, the present invention provides an X-ray CT apparatus wherein in the X-ray CT apparatus having the above construction, the pitch Ph of each of photodiodes lying in the channel and slice directions, of the photodiode array is less than or equal to 0.6 mm.

In the X-ray CT apparatus according to the eleventh aspect, high resolution photography can be performed as compared with the conventional example (in the case of the pitch of 1.0 mm or more) because the pitch Ph of each photodiode is 0.6 mm or less.

In a twelfth aspect, the present invention provides an X-ray CT apparatus wherein in the X-ray CT apparatus according to the eleventh aspect, X-ray focal point control means is provided which moves an X-ray focal point to acquire signals sent from the photodiodes with a first position as an X-ray focal point and next acquire signals sent from the photodiodes with a second position moved by a distance Δ in the channel direction from the first position as an X-ray focal point.

Since an X-ray beam is radially emitted from an X-ray focal point, a channel-direction width of an X-ray bundle in the vicinity (at the position of a subject) of the center of rotation results in about ½ of a channel-direction width of the X-ray bundle at a scintillator position.

Thus, in the X-ray CT apparatus according to the twelfth aspect, the collection of the signals at the X-ray focal points different from each other by the distance A as viewed in the channel direction is performed twice. Consequently, even if the regions for launching of the X-ray bundles into the scintillator are the same, it is possible to collect or acquire signals different in the channel-direction position of the X-ray bundle in the vicinity (subject's position) of the center of rotation. It is thus possible to enhance resolution in the channel direction.

Incidentally, the X-ray focal point control means is, for example, an electromagnetic deflection device or an electrostatic deflection device disposed between an electron gun and a target.

In a thirteenth aspect, the present invention provides an X-ray CT apparatus wherein in the X-ray CT apparatus according to the twelfth aspect, Ph/2<Δ≦Ph.

As described in the twelfth aspect, the channel-direction width of the X-ray bundle in the vicinity (at the position of the subject) of the center of rotation results in about ½ of the channel-direction width of the X-ray bundle at the scintillator position. However, an accurate channel-direction width of an X-ray bundle at an actual subject position depends on a geometrical arrangement of the X-ray focal points, the subject and the X-ray detector and varies according to the apparatus and subject. That is, the distance A over which the X-ray focal point is moved, varies according to the apparatus and subject.

Therefore, Ph/2≦Δ≦Ph was set in the X-ray CT apparatus according to the thirteenth aspect. Within this range, the distance may be adjusted in conformity to the apparatus and the subject.

In a fourteenth aspect, the present invention provides an X-ray CT apparatus wherein in the X-ray CT apparatus of the above construction, the photodiodes respectively have signal terminals on surfaces opposite to light-receiving surfaces.

Since the photodiodes having the signal terminals on the surfaces on the light-receiving surface side have heretofore been adopted, there is a need to provide wiring spaces on the light-receiving surface side. This could be a hindrance to high resolution.

Therefore, the photodiodes having the signal terminals on the surfaces opposite to the light-receiving surfaces have been adopted in the X-ray CT apparatus according to the fourteenth aspect. Thus, there is no need to provide the wiring spaces on the light-receiving surface side. This becomes effective for high resolution.

In a fifteenth aspect, the present invention provides an X-ray CT apparatus comprising an X-ray tube and an X-ray detector in which a scintillator is laminated on an upper surface of a photodiode array in which photodiodes are two-dimensionally arranged in channel and slice directions and the photodiodes adjacent to one another in the slice direction are arranged with being shifted in position by a ½ pitch in the channel direction.

In the X-ray CT apparatus according to the fifteenth aspect, a helical pitch is reduced and thereby approximately the same position of subject is shifted in the channel direction by the ½ pitch, whereby it can be photographed. Thus, the resolution in the channel direction can be enhanced twice.

In a sixteenth aspect, the present invention provides an X-ray CT apparatus comprising an X-ray tube and an X-ray detector in which a plurality of X-ray detector modules are arranged in a channel direction along a circular arc, wherein the ends in the channel direction, of the X-ray detector modules are formed as tapered surfaces in such a manner that the X-ray detector modules adjacent to one another are brought into close contact with one another.

Since each of the conventional X-ray detector modules was shaped in the form of rectangular parallelepiped, a triangle pole-like gap was defined between the adjacent X-ray detector modules when the plurality of X-ray detector modules were arranged in the channel direction along a circular arc.

In contrast, no triangle pole-like gap is defined in the X-ray CT apparatus according to the sixteenth aspect, and correspondingly the scintillator and photodiodes can be brought into larger size. It is thus possible to enhance the sensitivity of detection.

According to the X-ray CT apparatus of the present invention, high resolution photography can be carried out.

An X-ray CT apparatus of the present invention is used in high resolution photography.

Further objects and advantages of the present invention will be apparent from the following description of the preferred embodiments of the invention as illustrated in the accompanying drawings.

### BRIEF DESCRIPTION OF THE DRAWINGS

FIG. 1 is a configuration explanatory view showing an X-ray CT apparatus according to an embodiment 1.

FIG. 2 is a side view illustrating an X-ray detector module according to the embodiment 1.

FIG. 3 is a front view depicting the X-ray detector module according to the embodiment 1.

FIG. 4 is a bottom view showing the X-ray detector module according to the embodiment 1.

FIG. 5 is a top view illustrating the X-ray detector module according to the embodiment 1.

FIG. 6 is a front view showing a multidetector according to the embodiment 1.

FIG. 7 is a circuit diagram depicting a configuration of a signal transfer section according to the embodiment 1.

FIG. 8 is a circuit diagram showing another configuration of the signal transfer section according to the embodiment 1.

FIG. 9 is an explanatory view showing an X-ray bundle.

FIG. 10 is an explanatory view illustrating a state in which signals are collected at X-ray focal points Fa and Fb.

FIG. 11 is a side view showing a high-resolution X-ray detector module according to an embodiment 2.

FIG. 12 is a bottom view illustrating the high-resolution X-ray detector module according to the embodiment 2.

FIG. 13 is a top view depicting the high-resolution X-ray detector module according to the embodiment 2.

FIG. 14 is a side view showing a low-resolution X-ray detector module according to the embodiment 2.

FIG. 15 is a front view illustrating the low-resolution X-ray detector module according to the embodiment 2.

FIG. 16 is a bottom view depicting the low-resolution X-ray detector module according to the embodiment 2.

FIG. 17 is a top view showing the low-resolution X-ray detector module according to the embodiment 2.

FIG. 18 is a circuit diagram illustrating a configuration of a signal transfer section according to the embodiment 2.

FIG. 19 is a circuit diagram showing another configuration of the signal transfer section according to the embodiment 2.

FIG. 20 is a side view illustrating a multidetector according to an embodiment 3.

FIG. 21 is a front view depicting the multidetector according to the embodiment 3.

FIG. 22 is a bottom view showing the multidetector according to the embodiment 3.

FIG. 23 is a top view illustrating the multidetector according to the embodiment 3.

### DETAILED DESCRIPTION OF THE INVENTION

The present invention will hereinafter be described in further detail by illustrated embodiments. Incidentally, the present invention is not limited to the embodiments.

### Embodiment 1

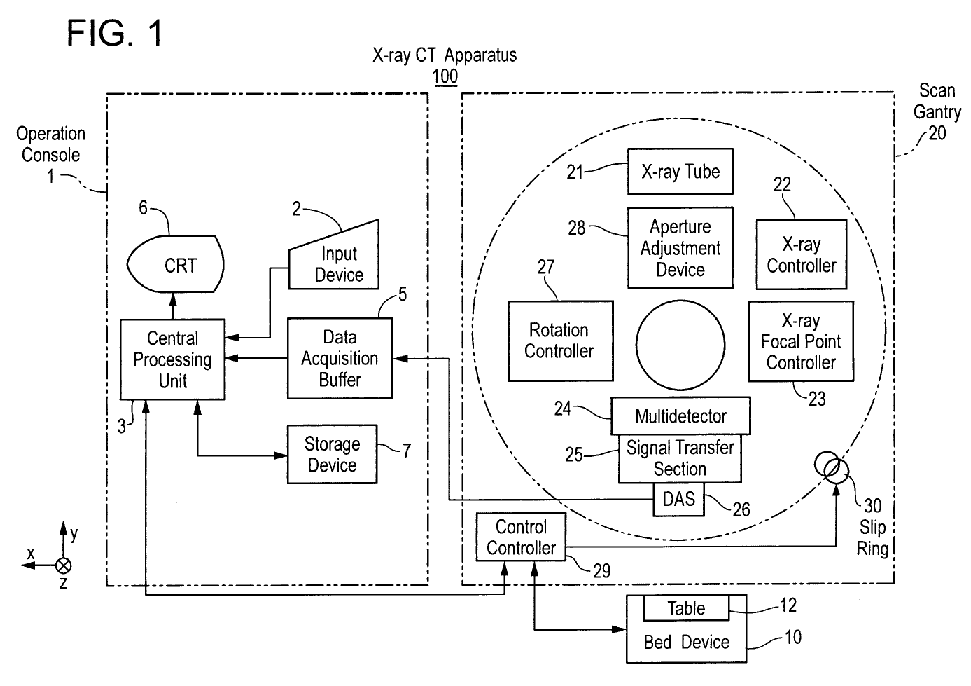

FIG. 1 is a plan view showing an X-ray CT apparatus 100 according to an 1.

The X-ray CT apparatus 100 is equipped with an operation console 1, a bed device 10 and a scan gantry 20.

The operation console 1 is equipped with an input device 2 which accepts an input from an operator, a central processing unit 3 which executes an image reconstructing process or the like, a data acquisition buffer 5 which acquires or collects projection data acquired by the scan gantry 20, a CRT 6 which displays a CT image reconstructed from the projection data, and a storage device 7 which stores programs, data and CT images therein.

The bed device 10 is provided with a table 12 which inserts and draws a subject into and from a bore (cavity portion) of the scan gantry 20 with the subject placed thereon. The table 12 is elevated (in a y-axis direction) and moved linearly (in a z-axis direction) by a motor built in the bed device 10.

The scan gantry 20 is equipped with an X-ray tube 21, an X-ray controller 22 which controls a tube voltage/tube current, an X-ray focal point controller 23 which controls the position of an X-ray focal point, an aperture adjustment device 28 which controls an aperture for controlling the spread of an X-ray beam, a multidetector 24 having a plurality of detector sequences, a signal transfer section 25 which transfers a signal outputted from the multidetector 24 to a DAS (Data Acquisition System) 26, the DAS 26, a rotation controller 27 which rotates the X-ray tube 21 or the like about the center of rotation (approximately equal to a body axis of the subject), a control controller 29 which performs a transfer of control signals from and to the operation console 1 and the bed device 10, and a slip ring 30.

The amount of a linear movement of the table 12 is counted by an encoder built in the bed device 10. The control controller 29 calculates a Z-axis coordinate of the table 12 from the amount of the linear movement and transmits the z-axis coordinate to the DAS 25 via the slip ring 30, followed by being added to the projection data.

The signal obtained at the multidetector 24 is AD-converted by the DAS 26 and transferred to the data acquisition buffer 5 via the slip ring 30 as projection data together with the z-axis coordinate.

The central processing unit 3 effects a pretreatment and an image reconstructing process on the projection data collected into the data acquisition buffer 5 to generate a CT image.

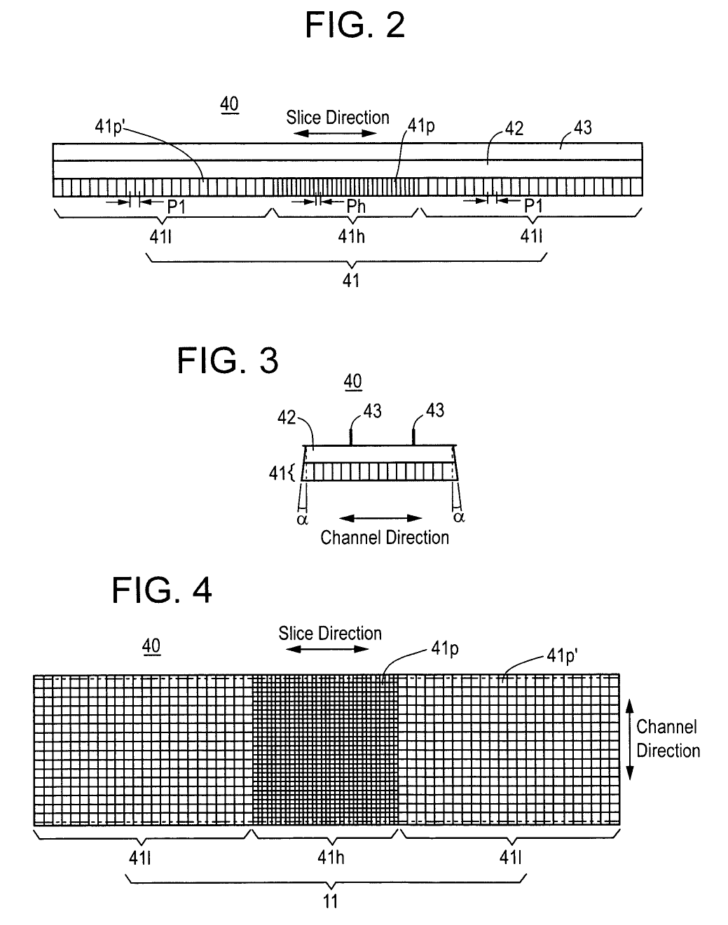

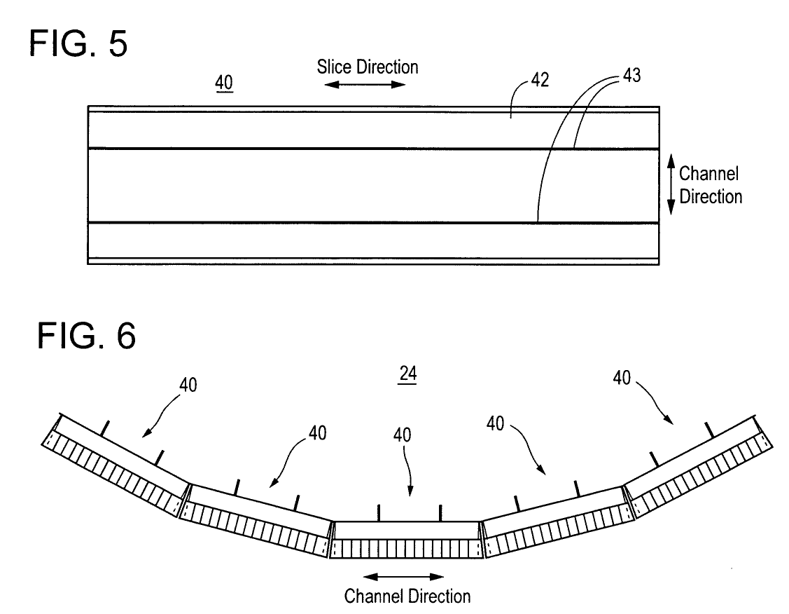

FIG. 2 is a side view of an X-ray detector module 40 according to the embodiment 1. FIG. 3 is a front view thereof. FIG. 4 is a bottom view thereof. FIG. 5 is a top view thereof.

The X-ray detector module 40 has a structure wherein a nonfractionated or nondivided scintillator 42 is laminated on an upper surface of a photodiode array 41 and collimators 43 extending in a slice direction (z-axis direction) at intervals of plural channel skips are placed on the scintillator 42. The X-ray detector module 40 does not have an X-ray shield extending in a channel direction.

The photodiode array 41 comprises a high resolution block 41h and low resolution blocks 41l which interpose the high resolution block 41h therebetween as viewed in the slice direction.

The high resolution block 41h is equivalent to one wherein photodiodes 41p are two-dimensionally arranged with a pitch Ph=0.5 mm (it is formed on one semiconductor substrate). The number of photodiodes as viewed in the channel direction is 32 and the number of photodiodes as viewed in the slice direction is 32.

Incidentally, since the ends of the X-ray detector module 40 as viewed in the channel direction have surfaces tapered at angles α as shown in FIG. 3, the photodiodes 41p at both ends as viewed in the channel direction become larger only slightly in the channel direction than others.

The angle α is equivalent to a fan angle÷the number of X-ray detector modules constituting the multidetector 24÷2. If, for example, the fan angle is 60° and the number of X-ray detector modules constituting the multidetector 24=60, then α=0.5°.

Each of the low resolution blocks 41l is equivalent to one wherein photodiodes 41p′ each of which is twice as large in size as each photodiode 41p of the high resolution block 41h as viewed in the channel and slice directions, are two-dimensionally arranged with a pitch Pl=2×Ph=1.0 mm (the low resolution blocks 41l are formed on one semiconductor substrate). The number of photodiodes as viewed in the channel direction is 16 and the number of photodiodes as viewed in the slice direction is 24.

Incidentally, since the ends of the X-ray detector module 40 as viewed in the channel direction have the surfaces tapered at the angles α as shown in FIG. 3, the photodiodes 41p′ at both ends as viewed in the channel direction become larger only slightly in the channel direction than others.

The scintillator 42 has no reflectors and slits. That is, it is a scintillator which has not been divided into cells and which is made up of a high-density material and has a thickness of 1 mm.

Each of the collimators 43 is a metal plate which extends in the slice direction. They are respectively placed between a fourth channel and a fifth channel as viewed from both ends as seen in the channel direction.

FIG. 6 is a front view of the multidetector 24.

The multidetector 24 is equivalent to one in which, for example, 60 X-ray detector modules 40 are arranged along a circular arc as viewed in the channel direction. Since the ends of each X-ray detector module 40 as viewed in the channel direction have the surfaces tapered at the angles α, the X-ray detector modules 40 adjacent to one another can be arranged so as to be brought into contact with one another. Since the photodiode array 41 of each X-ray detector module 40 is spread, the scintillator 42 and photodiodes 41p and 41p′ can be made large correspondingly. Thus, the sensitivity of detection can be enhanced.

The number of photodiodes as viewed in the channel direction in the high resolution blocks 41h used as the multidetector 24 is Ch=60×32=1820, and the number of photodiodes as viewed in the slice direction is Sh=32.

Also the number of photodiodes as viewed in the channel direction in the low resolution blocks 41l is Cl=60×16=960, and the number of photodiodes as viewed in the slice direction is Sl=2×24=48.

The signal transfer section 25 is capable of performing switching to the transfer of 61440 (=60×32×32) signals sent from the photodiodes 41p of the respective high resolution blocks 41h of the 64 X-ray detector modules 40 to the DAS 26 or the transfer of 15360 (=60×32×32/(2×2)) signals obtained by adding signals sent from the photodiodes 41p of the respective high resolution blocks 41h of the 60 X-ray detector modules 40 four by four (=2×2) and 46080 (=60×16×24×2) sent from the photodiodes 41p′ of the respective low resolution blocks 41l to the DAS 26.

The number D of signals inputtable to the DAS 26 reaches 61440.

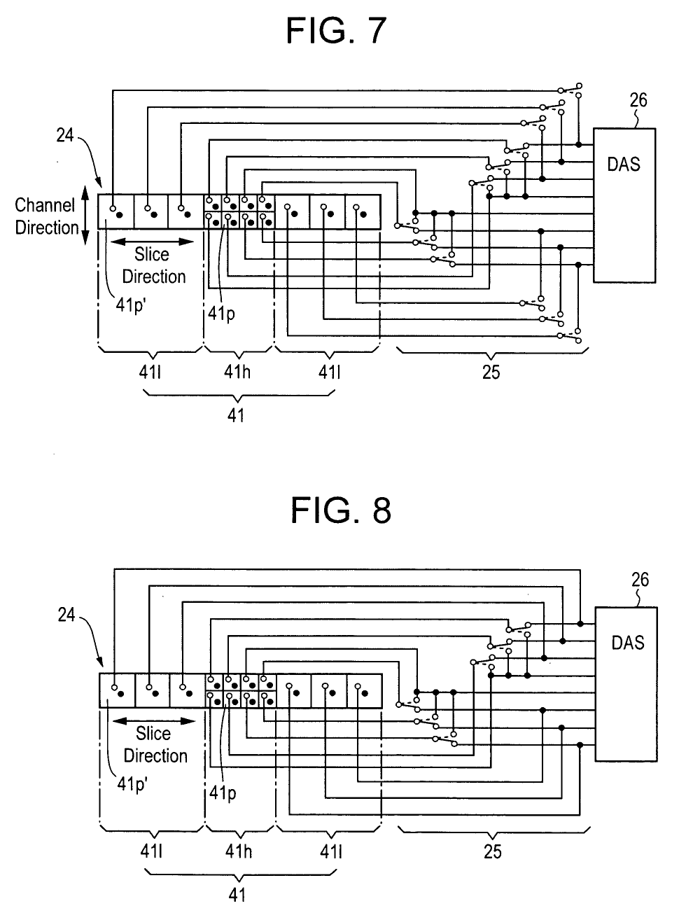

FIG. 7 is a typical diagram showing a configuration of the signal transfer section 25 according to the embodiment 1.

For simplification of explanation, the multidetector 24 is simplified as one having 2×4 photodiodes 41p contained in a high resolution block 41h and having 1×3 photodiodes 41p′ contained in each of low resolution blocks 411. Further, the DAS 26 is simplified as one which is capable of accepting eight signals.

Incidentally, white circles and black circles illustrated in the respective photodiodes 41p and 41p′ respectively indicate signal terminals respectively provided on surfaces opposite to light-detecting surfaces. The terminals indicated by the white circles show wirings in FIG. 7 as signal fetching or taken-out terminals. The terminals indicated by the black circles are used as common wiring terminals and their wirings are not shown in the drawing.

The signal transfer section 25 changes switches to positions indicated by solid lines or positions indicated by broken lines in FIG. 7.

In a state in which the switches are changed over to the positions indicated by the solid lines in FIG. 7, 8 (=2×4) signals sent from the photodiodes 41p of the high resolution block 41h are transferred to the DAS 26.

In a state in which the switches are changed over to the positions indicated by the broken lines in FIG. 7, 2 (=2×4/(2×2)) signals obtained by adding signals sent from the photodiodes 41p of the high resolution block 41h 4 (=2×2) by 4, and 6 (=1×3×2) signals sent from the photodiodes 41p′ of each low resolution block 41l are transferred to the DAS 26.

FIG. 8 is a typical diagram showing another configuration of the signal transfer section 25 according to the embodiment 1.

The signal transfer section 25 shown in FIG. 8 adds signals sent from the photodiode 41p′ of each low resolution block 41l in the signal transfer section 25 of FIG. 7 to signals sent from the photodiodes 41p of the corresponding high resolution block 41h by wired-ORing and transfers the result of addition to the DAS 26.

In such a configuration, there is a need to narrow an X-ray beam by the aperture adjustment device 28 so that the X-ray beam is launched into the high resolution block 41h alone upon high-resolution photography.

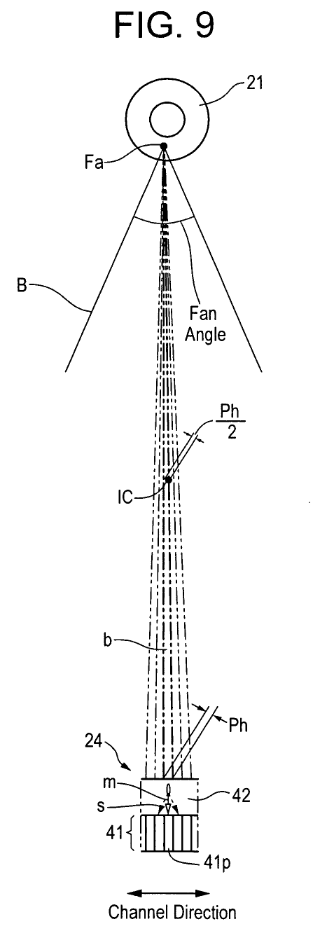

Since an X-ray beam B is radially emitted from an X-ray focal point Fa as shown in FIG. 9, a channel-direction width of an X-ray bundle b in the vicinity (at the position of a subject) of the center of rotation IC results in about ½ of a channel-direction width of the X-ray bundle at a scintillator position ≈ pitch Ph of a given photodiode 41p when the X-ray bundle b corresponding to the given photodiode 41p is taken into consideration. When the pitch of the photodiode 41p is Ph=0.5 mm, for example, the channel-direction width of the X-ray bundle b in the vicinity (at the subject's position) of the center of rotation IC becomes about 0.25 mm.

A white head arrow m shown in FIG. 9 indicates that light having the X-ray bundle b is launched into its corresponding photodiode 41p. When the thickness of the scintillator 42 is assumed to be 1 mm, the center of emission of the light having the X-ray bundle b is assumed to be located at a depth of 0.3 mm as viewed from the surface of the scintillator 42 and on the center line of the corresponding photodiode 41p, and the pitch of the photodiode 41p is Ph=0.5 mm, an incident angle θm is given as follows:
|θm|<19.7°

On the other hand, each of black head arrows s shown in FIG. 9 indicates that the light having the X-ray bundle b is launched into each photodiode adjacent to the corresponding photodiode 41p. When the thickness of the scintillator 42 is assumed to be 1 mm, the center of emission of the light having the X-ray bundle b is assumed to be located at the depth of 0.3 mm as viewed from the surface of the scintillator 42 and on the center line of the corresponding photodiode 41p, and the pitch of the photodiode 41p is Ph=0.5 mm, an incident angle θs is given as follows:
19.7<|θs|<47°

Thus, since the range of the incident angle widely varies, no problem occurs even if the light having the X-ray tube b is launched into each photodiode adjacent to the corresponding photodiode 41p.

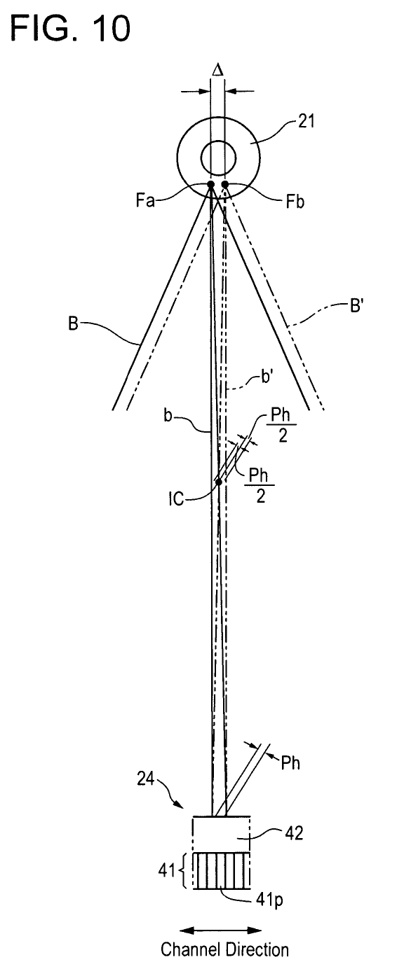

As shown in FIG. 10, the central processing unit 3 emits or applies an X-ray beam B via the X-ray focal point controller 23 with a first position as an X-ray focal point Fa to collect or acquire signals sent from photodiodes 41p.

Next, the central processing unit 3 applies an X-ray beam B via the X-ray focal point controller 23 with a second position moved by a distance A in the channel direction from the first position as an X-ray focal point Fb to collect signals sent from the photodiodes 41p.

The distance Δ is adjusted in conformity to the apparatus and the subject within a range of Ph/2≦Δ≦Ph. A channel-direction position at the X-ray focal point Fa, of the X-ray bundle b in the vicinity of the center of rotation IC (at the position of the subject) and a channel-direction position thereof at the X-ray focal point Fb are made different from each other by the width of the X-ray bundle b in the vicinity of the center of rotation IC (at the position of the subject).

It is thus possible to enhance resolution in the channel direction.

According to the X-ray CT apparatus 100 of the embodiment 1, the following advantageous effects are brought about.

(1) In an application requiring a high resolution image, the signals sent from the respective ones of the photodiodes 41p in the high resolution block 41h are transferred to the DAS 26. In an application enough at a low resolution image, the signals sent from the 2×2 photodiodes of the photodiodes 41p in the high resolution block 41h are added and transferred to the DAS 26, and the signals delivered from the respective ones of the photodiodes 41p′ in each low resolution block 41l are transferred to the DAS 26. Thus, it is possible to freely select high resolution photography and low resolution photography. Since the number of signals D is the same (D=Ch×Sh=Ch×Sh/(N×N)+Cl×Sl) even in the case of either the high resolution photography or the low resolution photography, the DAS 26 can be put to full use. It is possible to extend a photography range upon the low resolution photography.

(2) The scintillator 42 unfractionated into a large number of cells by the reflectors or slits or the like has been adopted. Thus, since there is no reduction in luminous or light-emission efficiency due to each of the reflectors or slits or the like, the pitch Ph of each photodiode 41p in the photodiode array 41 can be reduced to less than or equal to 0.6 mm.

(3) The scintillator 42 was thinned to less than or equal to 1 mm. Thus, it is possible to restrain the photodiodes 41p adjacent to each other from receiving light to be received by the given photodiode 41p.

(4) The collimators 43 extending on the scintillator 42 in the slice direction in the form of the plural channel skips have been adopted. Thus, since a reduction in luminous efficiency due to each of the collimators 43 can be suppressed, the pitch Ph of each photodiode 41p in the photodiode array 41 can be reduced to less than or equal to 0.6 mm.

(5) The X-ray shield extending on the scintillator 42 in the channel direction is not provided. Thus, since a reduction in luminous efficiency due to the X-ray shield can be suppressed, the pitch Ph of each photodiode 41p in the photodiode array 41 can be reduced to less than or equal to 0.6 mm.

(6) The collection of the signals at the X-ray focal points Fa and Fb different from each other by the distance Δ (Ph/2≦Δ≦Ph) as viewed in the channel direction is performed twice. It is thus possible to enhance resolution in the channel direction.

(7) The photodiodes 41p having the signal terminals on the surfaces opposite to the light-receiving surfaces have been adopted. Thus, there is no need to provide a wiring space on each light-receiving surface side. This becomes effective for high resolution.

(8) The ends in the channel direction, of the X-ray detector module 40 are shaped in the form of the surfaces tapered at the angles α. Thus, when the plurality of X-ray detector modules 40 are arranged along the circular arc in the channel direction, no triangle pole-like gap is defined between the X-ray detector modules 40 adjacent to each other, and they are adhered to each other. It is therefore possible to bring the scintillators 42 and the photodiodes 41p into large size and enhance the sensitivity of detection.

### Embodiment 2

An embodiment 2 is equipped with a high-resolution X-ray detector and a low-resolution X-ray detector in isolation as the multidetector 24.

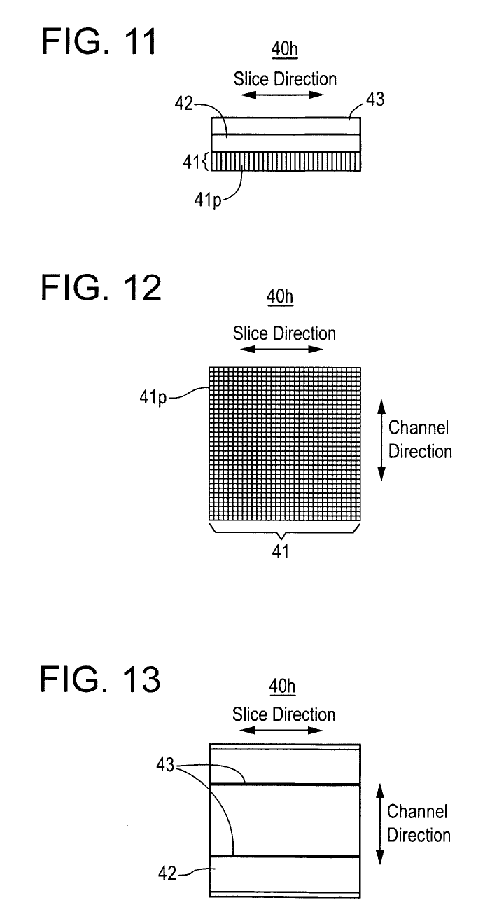

FIG. 11 is a side view of a high-resolution X-ray detector module 40h according to the embodiment 2. A front view thereof shown in the same figure is identical to one shown in FIG. 3. FIG. 12 is a bottom view thereof. FIG. 13 is a top view thereof.

The high-resolution X-ray detector module 40h has a structure wherein a nonfractionated or nondivided scintillator 42 is laminated on an upper surface of a photodiode array 41 and collimators 43 extending in a slice direction in the form of plural channel skips are placed on the scintillator 42. The high-resolution X-ray detector module 40h is not provided with an X-ray shield extending in a channel direction.

The photodiode array 41 is equivalent to one wherein photodiodes 41p are two-dimensionally arranged with a pitch Ph=0.5 mm (it is formed on one semiconductor substrate). The number of photodiodes as viewed in the channel direction is 32 and the number of photodiodes as viewed in the slice direction is 32.

Incidentally, since the ends of the high-resolution X-ray detector module 40h as viewed in the channel direction have surfaces tapered at angles a as shown in FIG. 3, the photodiodes 41p at both ends as viewed in the channel direction become larger only slightly in the channel direction than others. The angle α is 0.50.

The scintillator 42 has no reflectors and slits. That is, it is a scintillator which has not been divided into cells and which is made up of a high-density material and has a thickness of 1 mm.

Each of the collimators 43 is a metal plate which extends in the slice direction. They are respectively placed between a fourth channel and a fifth channel as viewed from both ends as seen in the channel direction.

In a manner similar to one shown in FIG. 6, 64 high-resolution X-ray detector modules 40h are arranged along a circular arc in the channel direction to configure a high-resolution X-ray detector.

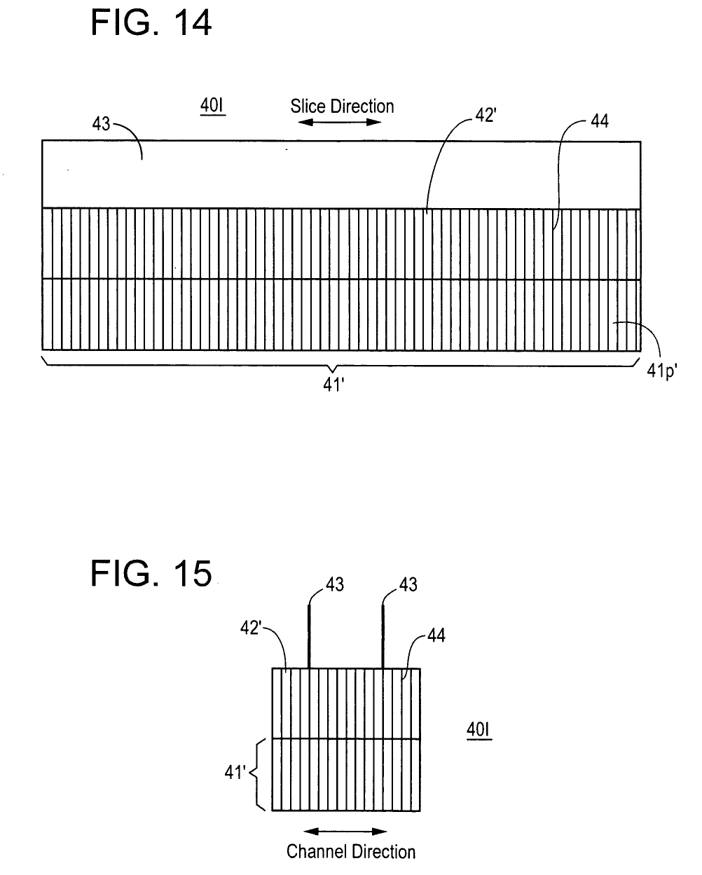

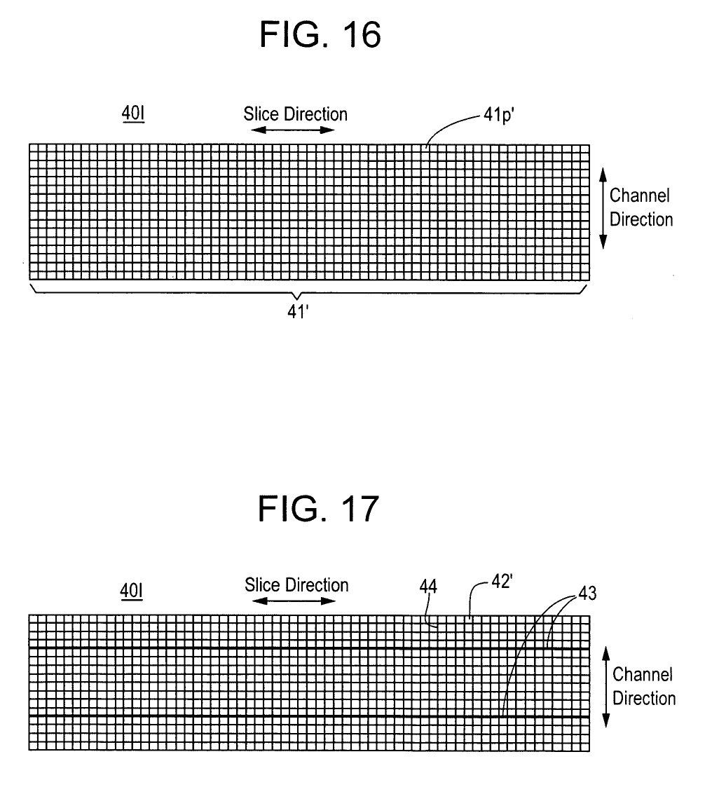

FIG. 14 is a side view of a low-resolution X-ray detector module 40l according to the embodiment 2. FIG. 15 is a front view thereof. FIG. 16 is a bottom view thereof. FIG. 17 is a top view thereof.

The low-resolution X-ray detector module 40l has a structure wherein scintillators 42′ fractionated or demarcated by reflectors 44 are laminated on an upper surface of a photodiode array 41′, and collimators 43 extending in a slice direction at intervals of plural channel skips are disposed on the scintillators 42′. The low-resolution X-ray detector module 40l is not provided with an X-ray shield extending in a channel direction.

The photodiode array 41′ is equivalent to one wherein photodiodes 41p′ each of which is twice as large in size as each photodiode 41p of the high-resolution X-ray detector as viewed in the channel and slice directions, are two-dimensionally arranged with a pitch Pl=2×Ph=1.0 mm (the photodiode array is formed on one semiconductor substrate). The number of photodiodes as viewed in the channel direction is 16 and the number of photodiodes as viewed in the slice direction is 32.

Each of the scintillators 42′ is a scintillator which has been divided into cells by the reflectors 44 and has a thickness of 4 mm.

Each of the collimators 43 is a metal plate which extends in the slice direction. They are respectively placed between a fourth channel and a fifth channel as viewed from both ends as seen in the channel direction.

In a manner similar to one shown in FIG. 6, 64 low-resolution X-ray detector modules 40l are arranged along a circular arc in the channel direction to configure a low-resolution X-ray detector.

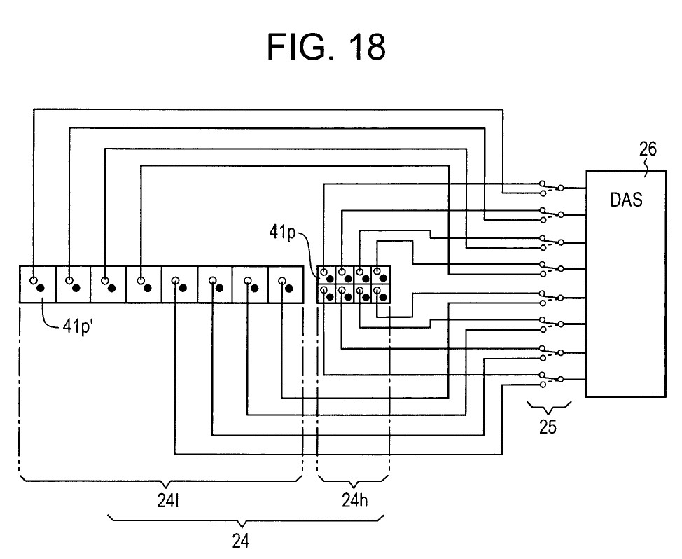

FIG. 18 is a typical diagram showing a configuration of a signal transfer section 25 according to the embodiment 2.

For simplification of explanation, the multidetector 24 is simplified as one having 2×4 photodiodes 41p contained in a high-resolution X-ray detector 24h and having 1×8 photodiodes 41p′ contained in a low-resolution X-ray detector 24l. Further, a DAS 26 is simplified as one which is capable of accepting eight signals.

Incidentally, white circles and black circles illustrated in the respective photodiodes 41p and 41p′ respectively indicate signal terminals respectively provided on surfaces opposite to light-detecting surfaces. The terminals indicated by the white circles show wirings in FIG. 18 as signal fetching or taken-out terminals. The terminals indicated by the black circles are used as common wiring terminals and their wirings are not shown in the drawing.

The signal transfer section 25 changes switches to positions indicated by solid lines or positions indicated by broken lines in FIG. 18.

In a state in which the switches are changed over to the positions indicated by the solid lines in FIG. 18, 8 (=2×4) signals delivered from the photodiodes 41p of the high-resolution X-ray detector 24h are transferred to the DAS 26.

In a state in which the switches are changed over to the positions indicated by the broken lines in FIG. 18, 8 (=1×8) signals sent from the photodiodes 41p′ of the low-resolution X-ray detector 24l are transferred to the DAS 26.

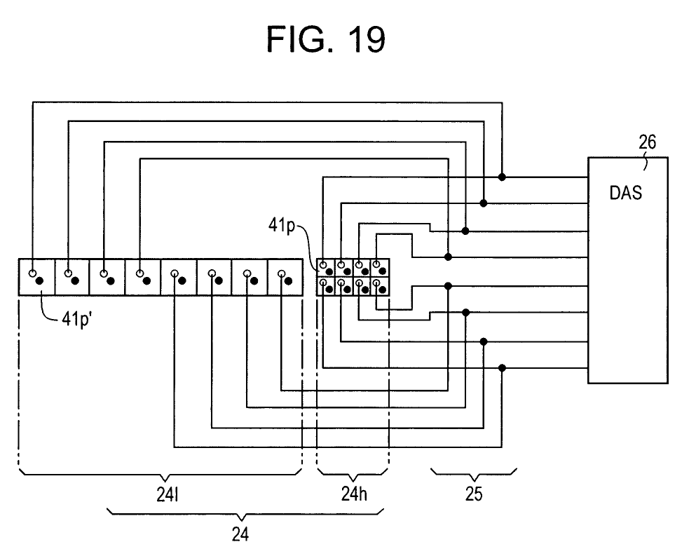

FIG. 19 is a typical diagram showing another configuration of the signal transfer section 25 according to the embodiment 2.

The signal transfer section 25 shown in FIG. 19 adds signals sent from photodiode 41p′ of a low-resolution X-ray detector 24l in the signal transfer section 25 of FIG. 18 to signals sent from photodiodes 41p of its corresponding high-resolution X-ray detector 24h by wired-ORing and transfers the result of addition to the DAS 26.

In the case of such a configuration, there is a need to perform switching of an X-ray beam by the aperture adjustment device 28 in such a manner that the X-ray beam is launched into the high-resolution X-ray detector 24h along upon high-resolution photography and the X-ray beam is launched into the low-resolution X-ray detector 24l alone upon low-resolution photography.

Even in the embodiment 2 in a manner similar to the embodiment 1, a central processing unit 3 emits or applies an X-ray beam B via an X-ray focal point controller 23 with a first position as an X-ray focal point Fa to collect or acquire signals sent from photodiodes 41p. Next, the central processing unit 3 applies an X-ray beam B via the X-ray focal point controller 23 with a second position moved by a distance A in the channel direction from the first position as an X-ray focal point Fb to collect signals sent from the photodiodes 41p.

The distance A is adjusted in conformity to the apparatus and the subject within a range of Ph/2≦Δ≦Ph.

It is thus possible to enhance resolution in the channel direction.

According to the X-ray CT apparatus of the embodiment 2, the following advantageous effects are brought about.

(1) In an application requiring a high resolution image, the signals from the photodiodes 41p in the high-resolution X-ray detector 24h are transferred to the DAS 26. In an application enough at a low resolution image, the signals sent from the photodiodes 41p′ in the low-resolution X-ray detector 24l are transferred to the DAS 26. Thus, it is possible to freely select high resolution photography and low resolution photography. Since the number of signals D is the same (D=Ch×Sh=Cl×Sl) even in the case of either the high resolution photography or the low resolution photography, the DAS 26 can be put to full use. It is possible to extend a photography range upon the low resolution photography.

(2) In the high-resolution X-ray detector 24h, the scintillator 42 unfractionated into a large number of cells by the reflectors or slits or the like has been adopted. Thus, since there is no reduction in luminous or light-emission efficiency due to each of the reflectors or slits or the like, the pitch Ph of each photodiode 41p in the photodiode array 41 can be reduced to less than or equal to 0.6 mm.

(3) The scintillator 42 was thinned to less than or equal to 1 mm in the high-resolution X-ray detector 24h. Thus, it is possible to restrain the photodiodes 41p adjacent to each other from receiving light to be received by the given photodiode 41p.

(4) The collimators 43 extending on the scintillator 42 in the slice direction in the form of the plural channel skips have been adopted. Thus, since a reduction in luminous efficiency due to each of the collimators 43 can be suppressed, the pitch Ph of each photodiode 41p in the photodiode array 41 can be reduced to less than or equal to 0.6 mm.

(5) The X-ray shield extending on the scintillator 42 in the channel direction is not provided. Thus, since a reduction in luminous efficiency due to the X-ray shield can be suppressed, the pitch Ph of each photodiode 41p in the photodiode array 41 can be reduced to less than or equal to 0.6 mm.

(6) The collection of the signals at the X-ray focal points Fa and Fb different from each other by the distance Δ (Ph/2≦Δ≦Ph) as viewed in the channel direction is performed twice. It is thus possible to enhance resolution in the channel direction.

(7) The photodiodes 41p having the signal terminals on the surfaces opposite to the light-receiving surfaces have been adopted. Thus, there is no need to provide a wiring space on each light-receiving surface side. This becomes effective for high resolution.

(8) The ends in the channel direction, of the high-resolution X-ray detector 40h are shaped in the form of the surfaces tapered at the angles a. Thus, when the plurality of high-resolution X-ray detector modules 40h are arranged along the circular arc in the channel direction, no triangle pole-like gap is defined between the high-resolution X-ray detector modules 40 adjacent to each other, and they are adhered to each other. It is therefore possible to bring the scintillators 42 and the photodiodes 41p into large size and enhance the sensitivity of detection.

### Embodiment 3

In an embodiment 3, a multidetector 24 is used in which photodiodes are arranged in zigzags.

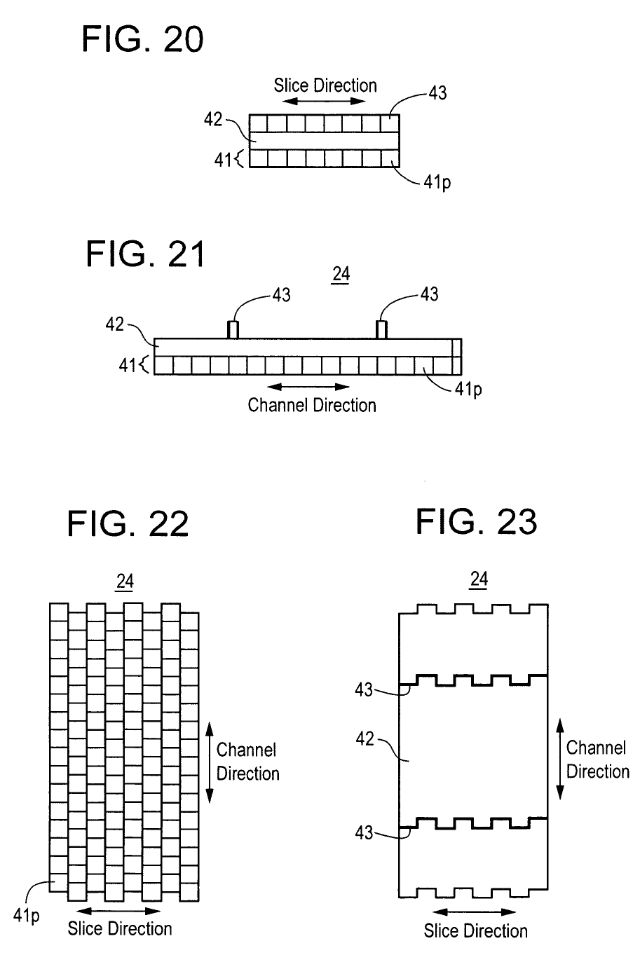

FIG. 20 is a side view of the multidetector 24 according to the embodiment 3. FIG. 21 is a front view thereof. FIG. 22 is a bottom view thereof. FIG. 23 is a top view thereof.

The multidetector 24 has a structure wherein an unfractionated scintillator 42 is laminated on an upper surface of a photodiode array 41, and collimators 43 extending in a slice direction in the form of plural channel skips are disposed on the scintillator 42. The multidetector 24 is not provided with an X-ray shield extending in a channel direction.

The photodiode array 41 is equivalent to one wherein photodiodes 41p are two-dimensionally arranged with a pitch Ph=0.5 mm (it is formed on one semiconductor substrate). However, the photodiodes 41p adjacent to one another in the slice direction are arranged with being shifted in position by a ½ pitch in the channel direction.

The scintillator 42 has no reflectors and slits. That is, it is a scintillator which has not been divided into cells and which is made up of a high-density material and has a thickness of 1 mm.

Each of the collimators 43 is a metal plate which extends in the slice direction. They are respectively placed between a fourth channel and a fifth channel as viewed from both ends as seen in the channel direction.

In the X-ray CT apparatus according to the embodiment 3, a helical pitch is reduced and thereby approximately the same position of subject is shifted in the channel direction by the ½ pitch, whereby it can be photographed. Thus, resolution in the channel direction can be enhanced twice.

Many widely different embodiments of the invention may be configured without departing from the spirit and the scope of the present invention. It should be understood that the present invention is not limited to the specific embodiments described in the specification, except as defined in the appended claims.

1. An X-ray CT apparatus comprising:
an X-ray tube;
an X-ray detector in which an unfractionated scintillator is laminated on an upper surface of a photodiode array comprising photodiodes two-dimensionally arranged in a channel direction and a slice direction;
a DAS which acquires signals delivered from the photodiodes; and
a signal switching device which switches whether to transfer the signals sent from the respective ones of the photodiodes to the DAS or to add the signals sent from the N×N (where N: integer greater than or equal to 2) photodiodes of the photodiodes and transfer the result of addition to the DAS.

2. The X-ray CT apparatus according to claim 1, wherein the photodiode array includes a high resolution block having a pitch extending in each of the channel and slice directions Ph<0.6 mm, and low resolution blocks each having a pitch extending in each of the channel and slice directions Pl=N×Ph, and when the number of the photodiodes in the channel direction in the high resolution block is Ch, the number of photodiodes in the slice direction is Sh, the number of the photodiodes in the channel direction in each of the low resolution blocks is Cl, the number of the photodiodes in the slice direction is Sl, and the number D of signals inputtable to the DAS is D, the following relationship is established:
D=Ch×Sh=Ch×Sh/(N×N)+Cl×Sl

3. An X-ray CT apparatus comprising:
an X-ray tube;
a high-resolution X-ray detector in which an unfractionated scintillator is laminated on an upper surface of a photodiode array comprising photodiodes two-dimensionally arranged with a pitch Ph<0.6 mm in each of channel and slice directions;
a low-resolution X-ray detector in which a scintillator is laminated on an upper surface of a photodiode array comprising photodiodes two-dimensionally arranged with a pitch Pl>Ph in the channel and slice directions;
a DAS which acquires signals from the photodiodes; and
a signal switching device which switches whether to transfer the signals from the photodiodes of the high-resolution X-ray detector to the DAS or to transfer the signals from the photodiodes of the low-resolution X-ray detector to the DAS.

4. An X-ray CT apparatus comprising:
an X-ray tube;
a high-resolution X-ray detector in which an unfractionated scintillator is laminated on an upper surface of a photodiode array comprising photodiodes two-dimensionally arranged with a pitch Pl<0.6 mm in channel and slice directions;
a low-resolution X-ray detector in which a scintillator is laminated on an upper surface of a photodiode array comprising photodiodes two-dimensionally arranged with a pitch Pl>Ph in the channel and slice directions;
a DAS which acquires signals from the photodiodes;
a signal adding device which adds the signals sent from the photodiodes of the high-resolution X-ray detector and the signals sent from the photodiodes of the low-resolution X-ray detector and transfers the result of addition to the DAS; and
an X-ray adjusting device which switches whether to launch an X-ray into the high-resolution X-ray detector alone or to launch an X-ray into the low-resolution X-ray detector alone.

5. The X-ray CT apparatus according to claim 3, herein when the number of the photodiodes lying in the channel direction in the high-resolution X-ray detector is Ch, the number of the photodiodes lying in the slice direction in the high-resolution X-ray detector is Sh, the number of the photodiodes lying in the channel direction in the low-resolution X-ray detector is Cl and the number of the photodiodes lying in the slice direction in the low-resolution X-ray detector is Sl, and the number of the signals inputtable to the DAS is D, the following relationship is established:
D=Ch×Sh=Cl×Sl

6. An X-ray CT apparatus comprising:
an X-ray tube; and
an X-ray detector in which a nonfractionated scintillator is laminated on an upper surface of a photodiode array comprising photodiodes two-dimensionally arranged in channel and slice directions.

7. The X-ray CT apparatus according to claim 6, wherein the thickness of the scintillator is less than or equal to 1 mm.

8. The X-ray CT apparatus according to claim 6, wherein the X-ray detector has collimators which extend in the slice direction on the scintillator at intervals of plural channel skips.

9. The X-ray CT apparatus according to claim 6, wherein the X-ray detector is not provided with an X-ray shield extending in the channel direction on the scintillator.

10. The X-ray CT apparatus according to claim 8, wherein the X-ray detector is not provided with an X-ray shield extending in the channel direction on the scintillator.

11. The X-ray CT apparatus according to claim 6, wherein the pitch Ph of each of photodiodes lying in the channel and slice directions, of the photodiode array is less than or equal to 0.6 mm.

12. The X-ray CT apparatus according to claim 11, further comprising an X-ray focal point control device which moves an X-ray focal point to acquire signals sent from the photodiodes with a first position as an X-ray focal point and next acquire signals sent from the photodiodes with a second position moved by a distance Δ in the channel direction from the first position as an X-ray focal point.

13. The X-ray CT apparatus according to claim 12, wherein Ph/2≦Δ≦Ph.

14. The X-ray CT apparatus according to claim 6, wherein the photodiodes respectively have signal terminals on surfaces opposite to light-receiving surfaces.

15. An X-ray CT apparatus comprising:
an X-ray tube; and
an X-ray detector in which a scintillator is laminated on an upper surface of a photodiode array in which photodiodes are two-dimensionally arranged in channel and slice directions and the photodiodes adjacent to one another in the slice direction are arranged with being shifted in position by a ½ pitch in the channel direction.

16. An X-ray CT apparatus comprising:
an X-ray tube; and
an X-ray detector in which a plurality of X-ray detector modules are arranged in a channel direction along a circular arc, wherein the ends in the channel direction, of the X-ray detector modules are formed as tapered surfaces in such a manner that the X-ray detector modules adjacent to one another are brought into close contact with one another.

17. The X-ray CT apparatus according to claim 4, wherein when the number of the photodiodes lying in the channel direction in the high-resolution X-ray detector is Ch, the number of the photodiodes lying in the slice direction in the high-resolution X-ray detector is Sh, the number of the photodiodes lying in the channel direction in the low-resolution X-ray detector is Cl and the number of the photodiodes lying in the slice direction in the low-resolution X-ray detector is Sl, and the number of the signals inputtable to the DAS is D, the following relationship is established:
D=Ch×Sh=Cl×Sl
